(19) United States
(12) Patent Application Publication
(10) Pub. No.: US 2026/0114738 A1
(43) Pub. Date: Apr. 30, 2026

(54) Automated Patient Management Using Remote Hemodynamic Monitoring

(71) Applicant: TC1 LLC, St. Paul, MN (US)

(72) Inventors: Andrew Siefert, Marietta, GA (US); Andrew Reiter, Minneapolis, MN (US); Megan Roberts, Cambridge, MA (US)

(73) Assignee: TC1 LLC, St. Paul, MN (US)

(21) Appl. No.: 19/003,848

(22) Filed: Dec. 27, 2024

Related U.S. Application Data
(60) Provisional application No. 63/616,018, filed on Dec. 29, 2023.

(51) Int. Cl. A61B 5/021; A61B 5/00; G16H 40/67

(52) U.S. Cl. CPC A61B 5/021; A61B 5/486; G16H 40/67

(57) ABSTRACT

Systems for automated patient management employ remote patient monitoring to trigger transmission of clinician defined interventional treatment instructions in response to satisfaction of clinician defined triggering criteria. A system for automated patient management execute a clinician portal module configured to receive pulmonary artery pressure data generated via a pulmonary artery pressure sensor, receive input defining a pulmonary artery pressure based interventional treatment triggering criteria, receive input defining interventional treatment instructions, process the pulmonary artery pressure data to determine whether the pulmonary artery pressure based interventional treatment triggering criteria is satisfied by the pulmonary artery pressure data, and in response to determining that the pulmonary artery pressure based interventional treatment triggering criteria is satisfied by the pulmonary artery pressure data, transmit the interventional treatment instructions to a patient mobile application.

### CROSS REFERENCE TO RELATED APPLICATION DATA

The present application claims the benefit of priority of U.S. Provisional Application No. 63/616,018 filed Dec. 29, 2023, the disclosure which is incorporated herein by reference in its entirety for all purposes.

### BACKGROUND

According to the World Health Organization, cardiovascular diseases (CVDs) are the leading cause of death worldwide. In 2019, it is estimated that 17.9 million people died from CVDs (which is 32% of all deaths worldwide). 85% of the CVD caused deaths in 2019 were attributed to heart attack and stroke. In view of the extent and impact of CVDs, improved approaches for effective and efficient evaluation and treatment of patients suffering from CVD are of great interest.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Embodiments described herein are directed to automated management of patients suffering from cardiovascular disease (CVD). In many embodiments, the pulmonary artery pressure of each patient is monitored via an implanted pulmonary artery pressure (PAP) sensor. For each patient, PAP data generated via the PAP sensor is transmitted to and stored in a centralized database. A patient management system is used to process the PAP data to monitor the patients for changes in condition for which changes in treatment protocol are indicated. In many embodiments, the patient management system is configured to execute a clinician portal module configured for obtaining pulmonary artery pressure based interventional treatment triggering criteria from a clinician via a clinician portal and corresponding interventional treatment instructions that are automatically transmitted to the patient in response to satisfaction of the interventional treatment triggering criteria. The automated monitoring of the PAP data combined with the automated transmission of the interventional treatment instructions reduce the workload on the clinician and reduce delay in revising treatment of the patient to address the changed patient condition.

Thus, in one aspect, a system for automated patient management includes at least one processor and at least one memory device storing non-transient instructions executable by the at least one processor to cause the at least one processor to execute a clinician portal module. The clinician portal module is configured to (a) receive pulmonary artery pressure data generated via a pulmonary artery pressure sensor implanted in a patient; (b) receive input via a clinician portal defining a pulmonary artery pressure based interventional treatment triggering criteria for the patient; (c) receive input, via the clinician portal, defining interventional treatment instructions to be transmitted to the patient in response to the pulmonary artery pressure data satisfying the pulmonary artery pressure based interventional treatment triggering criteria; (d) process the pulmonary artery pressure data to determine whether the pulmonary artery pressure based interventional treatment triggering criteria is satisfied by the pulmonary artery pressure data; and (e) in response to determining that the pulmonary artery pressure based interventional treatment triggering criteria is satisfied by the pulmonary artery pressure data, transmit the interventional treatment instructions to a patient mobile application associated with the patient.

In many embodiments of the system for automated patient management, the pulmonary artery pressure based interventional treatment triggering criteria is based on exceedance of one or more clinician defined limits. For example, the pulmonary artery pressure based interventional treatment triggering criteria can be based on one or more exceedances of an upper limit for a pulmonary artery pressure parameter for the patient. The pulmonary artery pressure based interventional treatment triggering criteria can be based on one or more instances of a pulmonary artery pressure parameter for the patient being less than a lower limit for the patient. In many embodiments, the pulmonary artery pressure based interventional treatment triggering criteria defines an upper limit and a lower limit for a pulmonary artery pressure parameter for the patient, the interventional treatment instructions include high-pressure interventional treatment instructions and low-pressure interventional treatment instructions, the high-pressure interventional treatment instructions are transmitted to the patient mobile application in response to a designated number of exceedances of the upper limit and the low-pressure interventional treatment instructions are transmitted to the patient mobile application in response to a designated number of occurrences of the pulmonary artery pressure parameter being less than the lower limit.

In many embodiments of the system for automated patient management, the interventional treatment instructions vary based on the number of exceedances of the clinician defined limit. For example, the pulmonary artery pressure based interventional treatment triggering criteria can define an upper limit for a pulmonary artery pressure parameter for the patient. The interventional treatment instructions can include first high-pressure interventional treatment instructions and second high-pressure interventional treatment instructions. The first high-pressure interventional treatment instructions can be transmitted to the patient mobile application in response to a first number of consecutive exceedances of the upper limit. The second high-pressure interventional treatment instructions can be transmitted to the patient mobile application in response to a second number of consecutive exceedances of the upper limit. The second number of consecutive exceedances of the upper limit can be greater than the first number of consecutive exceedances of the upper limit. In some embodiments of the system for automated patient management, the interventional treatment instructions further include third high-pressure interventional treatment instructions. The third high-pressure interventional treatment instructions can be transmitted to the patient mobile application in response to a third number of consecutive exceedances of the upper limit. The third number of consecutive exceedances of the upper limit can be greater than the second number of consecutive exceedances of the upper limit. The pulmonary artery pressure based interventional treatment triggering criteria can define a lower limit for a pulmonary artery pressure parameter for the patient. The interventional treatment instructions can include first low-pressure interventional treatment instructions and second low-pressure interventional treatment instructions. The first low-pressure interventional treatment instructions can be transmitted to the patient mobile application in response to a first number of consecutive instances of the pulmonary artery pressure parameter being less than the lower limit. The second low-pressure interventional treatment instructions can be transmitted to the patient mobile application in response to a second number of consecutive instances of the pulmonary artery pressure parameter being less than the lower limit. The second number of consecutive instances of the pulmonary artery pressure parameter can be less than the lower limit is greater than the first number of consecutive instances of the pulmonary artery pressure parameter being less than the lower limit. In some embodiments of the system for automated patient management, the interventional treatment instructions can further include third high-pressure interventional treatment instructions and third low-pressure interventional treatment instructions. The third low-pressure interventional treatment instructions can be transmitted to the patient mobile application in response to a third number of consecutive instances of the pulmonary artery pressure parameter being less than the lower limit. The third number of consecutive instances of the pulmonary artery pressure parameter can be less than the lower limit is greater than the second number of consecutive instances of the pulmonary artery pressure parameter being less than the lower limit.

The clinician module can be further configured for providing base-line treatment instructions to the patient. For example, the clinician portal module can be further configured to receive input via the clinician portal defining base-line treatment instructions for the patient and transmit the base-line treatment instructions to the patient mobile application. The base-line treatment instructions can define base-line medications for the patient.

In many embodiments of the system for automated patient management, the clinician module is further configured to provide for communication between the patient and the clinician. For example, the interventional treatment instructions can include a message for the patient.

In many embodiments of the system for automated patient management, the clinician portal module is configured to notify the clinician when the interventional treatment instructions are transmitted. For example, the clinician portal module can be further configured to, in response to transmitting the interventional treatment instructions to the patient mobile application, display an indication on the clinician portal that the interventional treatment instructions were transmitted to the patient mobile application.

In many embodiments of the system for automated patient management, the clinician is notified that the patient acknowledged receipt of the interventional instructions. For example, the clinician module can be further configured to receive an acknowledgement from the patient mobile application that the patient mobile application received an input indicating acknowledgement of receipt of the interventional treatment instructions and output an indication of the acknowledgement via the clinician portal.

In many embodiments of the system for automated patient management, the clinician can define when to be flag a patient for assessment by the clinician based on the pulmonary artery pressure of the patient. For example, the clinician portal module can be further configured to receive input via the clinician portal defining a pulmonary artery pressure based notification triggering criteria for the patient; process the pulmonary artery pressure data to determine whether the pulmonary artery pressure based notification triggering criteria is satisfied by the pulmonary artery pressure data; and in response to determining that the pulmonary artery pressure based notification triggering criteria is satisfied by the pulmonary artery pressure data, output a notification via the clinician portal that designates the patient for assessment.

The pulmonary artery pressure based notification triggering criteria can be based on the pulmonary artery pressure of the patient using any suitable approach. For example, the pulmonary artery pressure based notification triggering criteria can be based on one or more exceedances of a notification upper limit for a pulmonary artery pressure parameter for the patient. The pulmonary artery pressure based notification triggering criteria can be based on one or more instances of a pulmonary artery pressure parameter for the patient being less than a notification lower limit for the patient.

In many embodiments of the system for automated patient management, the patient mobile application has a number of pages for providing information to the patient and/or receiving input from the patient. For example, the patient mobile application can be configured for displaying a treatment change notification page that includes the interventional treatment instructions. The interventional treatment instructions can include a revised medication prescription. The interventional treatment instructions can include a message for the patient that was received via the clinician portal. The treatment change notification page can include an acknowledgement input button for acknowledging receipt of the interventional treatment instructions. The patient mobile application can be configured for displaying a medication prescription change notification page that includes an updated medication prescription. The patient mobile application can be configured to display a status page that includes a reading status indicator of accomplishment of prescribed readings of the pulmonary artery pressure sensor. The reading status indicator can include a reading status icon with a variable appearance for communicating a level of accomplishment of the prescribed readings of the pulmonary artery pressure sensor. The reading status icon can have a heart shape. The patient mobile application can be configured to display a status page that includes a lifestyle status indicator of accomplishment of prescribed lifestyle actions by the patient. The lifestyle status indicator can include a lifestyle status icon with a variable appearance for communicating a level of accomplishment of the prescribed lifestyle actions by the patient. The lifestyle status icon can have a heart shape. The patient mobile application can be configured to display a status page that includes a medication status indicator of accomplishment of taking prescribed medications by the patient. The medication status indicator can include a medication status icon with a variable appearance for communicating a level of accomplishment of taking the prescribed medications by the patient. The medication status icon can have a heart shape. The patient mobile application can be configured to display a status page that includes a pulmonary artery pressure reading section and display one of a reminder to accomplish a reading of the pulmonary artery pressure sensor, a plot of recent pulmonary artery pressures of the patient, or a notification regarding recent pulmonary artery pressures of the patient in the pulmonary artery pressure reading section. The patient mobile application can be configured to display a status page that includes a systemic blood pressure reading section and display one of a reminder to measure and enter systemic blood pressure of the patient or an indication of a systemic blood pressure of the patient in the systemic blood pressure reading section. The patient mobile application can be configured to display a readings page that includes a pulmonary artery pressure reading section; display a selection button for a day, a selection button for a week, and a selection button for a month; display a pulmonary artery pressure of the patient for a day when the selection button for a day is selected; display pulmonary artery pressures of the patient for a week when the selection button for a week is selected; and display pulmonary artery pressures of the patient for a month when the selection button for a month is selected. The patient mobile application can be configured to display a readings page that includes a systemic blood pressure reading section; display a selection button for a day, a selection button for a week, and a selection button for a month; display a systemic blood pressure reading of the patient for a day when the selection button for a day is selected; display systemic blood pressure readings of the patient for a week when the selection button for a week is selected; and display systemic blood pressure readings of the patient for a month when the selection button for a month is selected.

For a fuller understanding of the nature and advantages of the present invention, reference should be made to the ensuing detailed description and accompanying drawings.

### BRIEF DESCRIPTION OF THE DRAWINGS

FIG. 1 shows a simplified schematic diagram of a system for automated patient management using remote hemodynamic monitoring, in accordance with embodiments.

FIG. 2 illustrates a process in which clinical team members are employed to generate and transmit treatment instructions that can be employed in the system of FIG. 1.

FIG. 3 illustrates a process in which treatment instructions are automatically generated and transmitted that can be accomplished by the system of FIG. 1.

FIG. 4 shows a user interface for a treatment plan template for defining treatment instructions to be automatically transmitted to a patient and criteria for triggering transmission of the treatment instructions based on monitored pulmonary artery pressure of the patient.

FIG. 5 illustrates entry of an optimal range of diastolic pulmonary artery pressure (PAP diastolic) for a patient into the treatment plan template of FIG. 4.

FIG. 6 shows user interface input buttons of the user interface for the treatment plan template of FIG. 4 that can be selected to access entry forms for specifying daily medications for the patient and for specifying treatment instructions to be automatically transmitted to the patient in response to the pulmonary artery pressure of the patient satisfying defined triggering criteria.

FIG. 7 shows a daily medication entry form for the treatment plan template of FIG. 4.

FIG. 8 illustrates display of specified daily medications for a patient in the treatment plan template of FIG. 4.

FIG. 9 shows an intervention instruction entry selection form for the treatment plan template of FIG. 4.

FIG. 10 and FIG. 11 show intervention instruction entry forms for the treatment plan template of FIG. 4.

FIG. 12 illustrates display of specified daily medications and intervention instructions for the patient in the treatment plan template of FIG. 4.

FIG. 13 shows user interface input buttons of the treatment plan template of FIG. 4 that can be selected to save or cancel the specified automated treatment plan for the patient.

FIG. 14 shows a confirmation screen that is displayed in response to selection of the cancel input button of FIG. 13.

FIG. 15 shows a notification screen that can be employed in the system of FIG. 1 in which the notification screen includes a list of patients designated as needing attention based on clinic defined settings.

FIG. 16 shows a notification screen that can be employed in the system of FIG. 1 in which the notification screen includes a list of patients designated as needing attention, a list of patients designated as belonging to an optimization phase, and a list of patient designated as part of a maintenance phase.

FIG. 17 shows a quick overview screen for a patient selected from the attention need section of the notifications page.

FIG. 18, FIG. 19, and FIG. 20 show example home pages of the patient mobile application of the system of FIG. 1.

FIG. 21, FIG. 22, FIG. 23, and FIG. 24 show example readings pages for manual and/or automated entry of systemic blood pressure for the patient mobile application of the system of FIG. 1.

FIG. 25, FIG. 26, FIG. 27, and FIG. 28 show example medications pages for the patient mobile application of the system of FIG. 1.

FIG. 29, FIG. 30, and FIG. 31 show example lifestyle pages for the patient mobile application of the system of FIG. 1.

FIG. 32, FIG. 33, and FIG. 34 show example messaging and notification pages for the patient mobile application of the system of FIG. 1.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

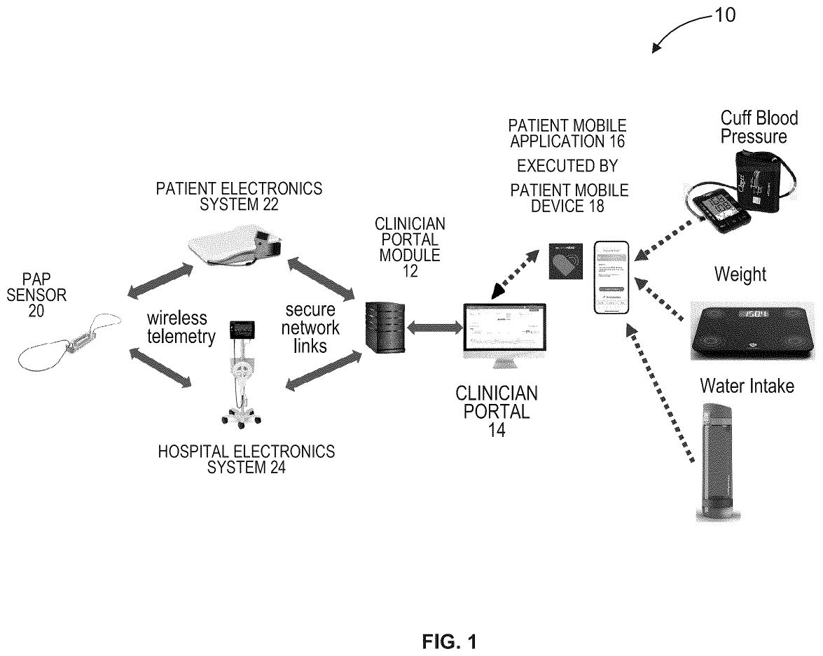

Turning now to the drawings in which similar reference identifiers refer to similar elements in the various figures, FIG. 1 shows a simplified schematic diagram of a system 10 for automated patient management using remote hemodynamic monitoring, in accordance with embodiments. The system 10 includes a clinician portal module 12, a clinician portal 14, a patient mobile application 16, a mobile device 18, an implanted pulmonary artery pressure (PAP) sensor 20, a patient electronics system 22 or a hospital electronics system 24, and a blood pressure measurement device 26 configured for non-invasive measurement of systemic blood pressure.

In many embodiments, the clinician module 12 is configured for providing automated patient management based on pulmonary artery pressure of the patient monitored via the PAP sensor 20. The clinician module 12 can be executed on by any suitable computing system, which can include at least one processor and at least one memory device storing non-transient instructions executable by the at least one processor to cause the at least one processor to execute the clinician portal module 12. For example, the clinician module 12 can be executed by one or more servers or by a computing system that includes the clinician portal 14. The clinician portal 14 can include one or more suitable display devices and one or more suitable input devices such as one or more keyboards. In many embodiments, the clinician module 12 is configured to receive and store pulmonary artery pressure data generated by the PAP sensor 20. While only one PAP sensor 20 is shown in FIG. 1, in many embodiments the clinician module 12 is configured to provide automated patient management for many different patients. And while only one clinician portal 14 is shown in FIG. 1, in many embodiments, the system 10 includes a number of different clinician portals 14, each of which can be located and operated by a different clinic to provide automated patient management for corresponding different sets of patients. In many embodiments, the clinician module 12 is configured to: (a) receive pulmonary artery pressure data generate via the PAP sensor 20, (b) receive input via the clinician portal 14 that defining a pulmonary artery pressure based interventional treatment triggering criteria for the patient, (c) receive input, via the clinician portal 14, defining interventional treatment instructions to be transmitted to the patient in response to the pulmonary artery pressure data satisfying the pulmonary artery pressure based interventional treatment triggering criteria, (d) process the pulmonary artery pressure data to determine whether the pulmonary artery pressure based interventional treatment triggering criteria is satisfied by the pulmonary artery pressure data, and (e) in response to determining that the pulmonary artery pressure based interventional treatment triggering criteria is satisfied by the pulmonary artery pressure data, transmit the interventional treatment instructions to the patient mobile application 16.

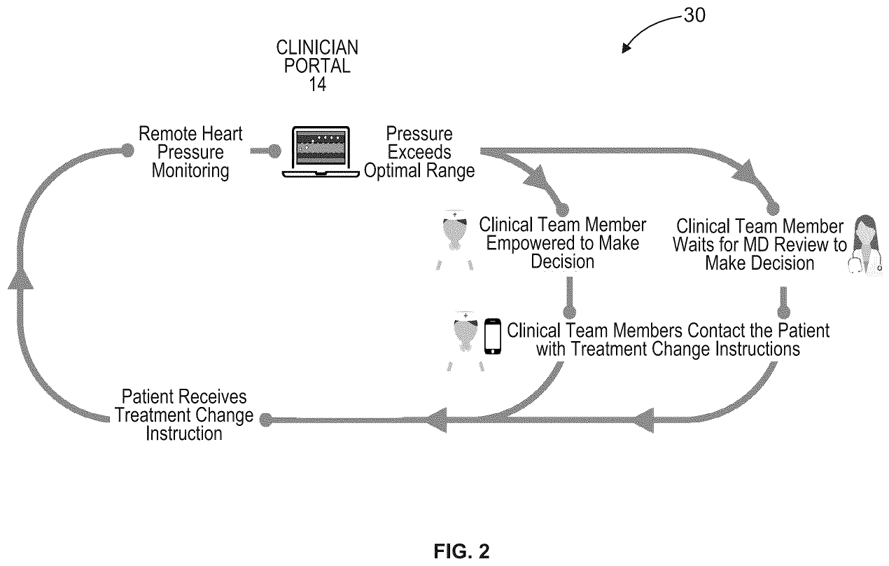

FIG. 2 illustrates a manual process 30 for patient management that can be accomplished using the system 10. In the process 30, clinical team members are employed to generate and transmit treatment instructions to the patient via the patient mobile application 16. In the process 30, the system 10 can flag a patient for further assessment by the clinical team members in response to a pulmonary artery pressure parameter of the patient exceeding an upper limit for the patient or being less than a lower limit for the patient. If the reviewing clinical team member is empowered to make a treatment decision, the clinical team member can define interventional treatment instructions, which can then be transmitted to the patient via the patient mobile application 16. Otherwise, the interventional treatment instructions can be defined by a suitable medical doctor and then transmitted to the patient via the patient mobile application 16.

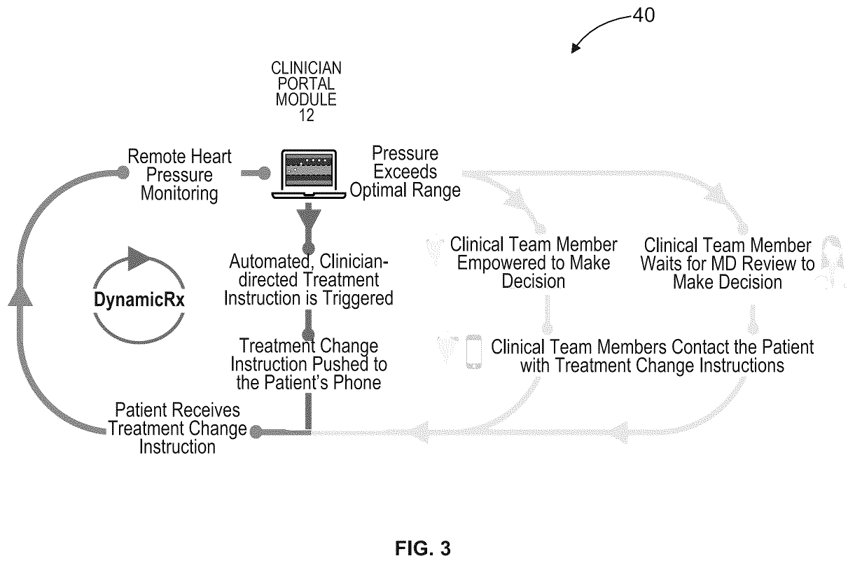

In contrast to the manual process 30, FIG. 3 illustrates an automated process 40 in which interventional treatment instructions are automatically transmitted to the patient mobile application 16 as described herein. In the automated process 40, interventional treatment instructions and criteria for triggering transmission of the interventional treatment instructions are preemptively defined by the clinician. The clinician portal module 12 automatically transmits the interventional treatment instructions to the patient in response to satisfaction of the criteria for triggering transmission of the interventional treatment instructions by the PAP data for the patient.

### Clinician Portal

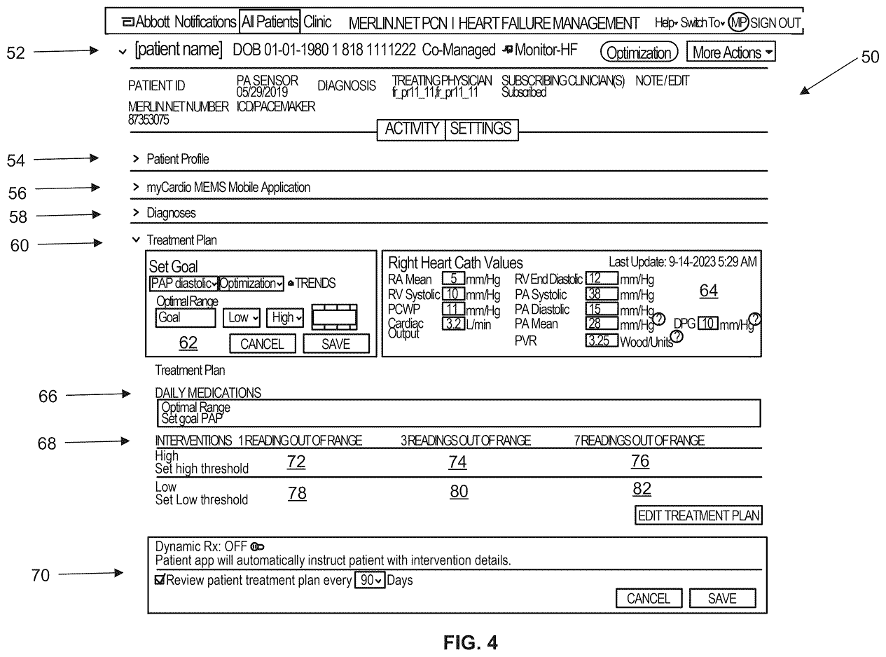

FIG. 4 through FIG. 17 show example user interface screens of the clinician portal 14. In many embodiments, each of the user interface screens of the clinician portal 14 are displayed on a display at a clinic by the clinician module 12.

FIG. 4 shows a user interface screen 50 for a managing a selected patient 52. The screen 50 displays associated information for the selected patient (e.g., patient ID, implantation date of the PAP sensor 20, treating physician, whether the patient has an implanted cardiac device or pacemaker). The screen 50 includes selectively expandable sections for patient profile 54, a section 56 for information on the patient's patient mobile application 16, patient diagnosis, and a treatment plan section 60 for defining an automated treatment plan for the patient. The treatment plan section 60 (which is shown expanded in FIG. 4) includes a PAP parameter section 62, a right heart catheterization values section 64, a daily medications section 66, an interventional treatment instructions section 68, and an interventional treatment instructions control section 70. The PAP parameters section 62 includes selectable input boxes for specifying a PAP parameter to monitor and for setting an optimal range for the PAP parameter. The right heart catheterization values section 64 displays blood pressure values for the patient obtained during the most recent right heart catheterization of the patient and the corresponding date of the most recent right heart catheterization of the patient. The daily medications section 66 is configured for selection and display of daily medications for the patient for usage while the selected PAP parameter is within the defined optimal range. The interventional treatment instructions section 68 is configured for selection and display of interventional treatment instructions to be automatically transmitted to the patient in response to the monitored PAP parameter satisfying the corresponding criteria for triggering transmission of the interventional treatment instructions based on monitored PAP parameter. In the illustrated embodiment, the interventional treatment instructions section 68 includes selectable inputs 72, 74, 76, 78, 80, 82 that can be employed or not employed in any suitable combination. Selectable input 72 is configured for entry of interventional treatment instructions to be automatically transmitted to the patient's mobile application 16 in response to one reading of the PAP parameter exceeding the upper limit for the PAP parameter defined in the PAP parameter section 62. Selectable input 74 is configured for entry of interventional treatment instructions to be automatically transmitted to the patient's mobile application 16 in response to three consecutive readings of the PAP parameter exceeding the upper limit for the PAP parameter defined in the PAP parameter section 62. Selectable input 76 is configured for entry of interventional treatment instructions to be automatically transmitted to the patient's mobile application 16 in response to seven consecutive readings of the PAP parameter exceeding the upper limit for the PAP parameter defined in the PAP parameter section 62. Selectable input 78 is configured for entry of interventional treatment instructions to be automatically transmitted to the patient's mobile application 16 in response to one reading of the PAP parameter being lower than the lower limit for the PAP parameter defined in the PAP parameter section 62. Selectable input 80 is configured for entry of interventional treatment instructions to be automatically transmitted to the patient's mobile application 16 in response to three consecutive readings of the PAP parameter being lower than the lower limit for the PAP parameter defined in the PAP parameter section 62. Selectable input 82 is configured for entry of interventional treatment instructions to be automatically transmitted to the patient's mobile application 16 in response to seven consecutive readings of the PAP parameter being lower than the lower limit for the PAP parameter defined in the PAP parameter section 62. The interventional treatment instructions control section 70 includes a selectable input for turning automatic transmission of interventional instructions to the patient on and off, as well as an input for selecting and defining an interval for reviewing the treatment plan for the patient.

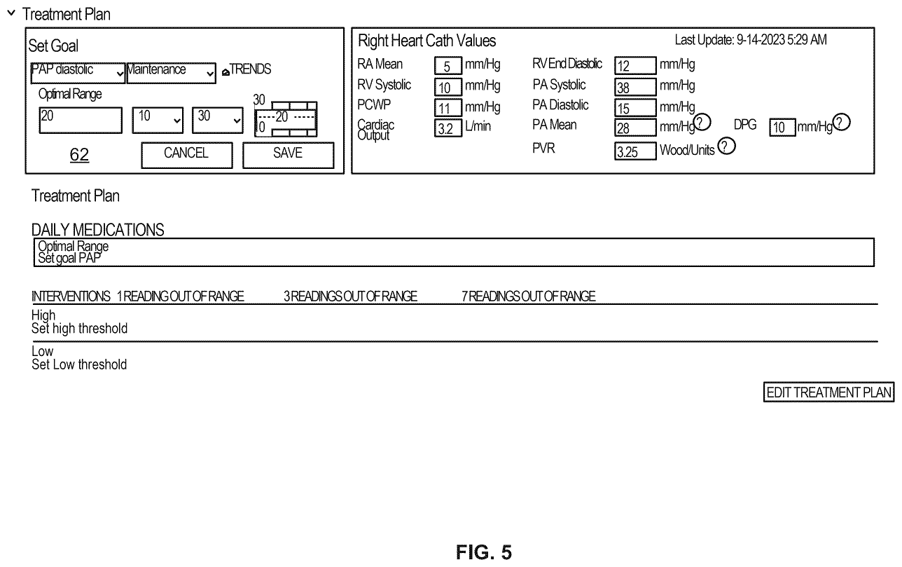

FIG. 5 illustrates entry of the PAP parameter to be monitored and the corresponding optimal range for the selected PAP parameter to be monitored in the PAP parameter section 62. In the example shown, PAP diastolic has been selected as the PAP parameter to be monitored, the categorization of the monitoring of the PAP parameter has been selected as maintenance, and the optimal range has been defined to be from 10 mmHg to 30 mmHg.

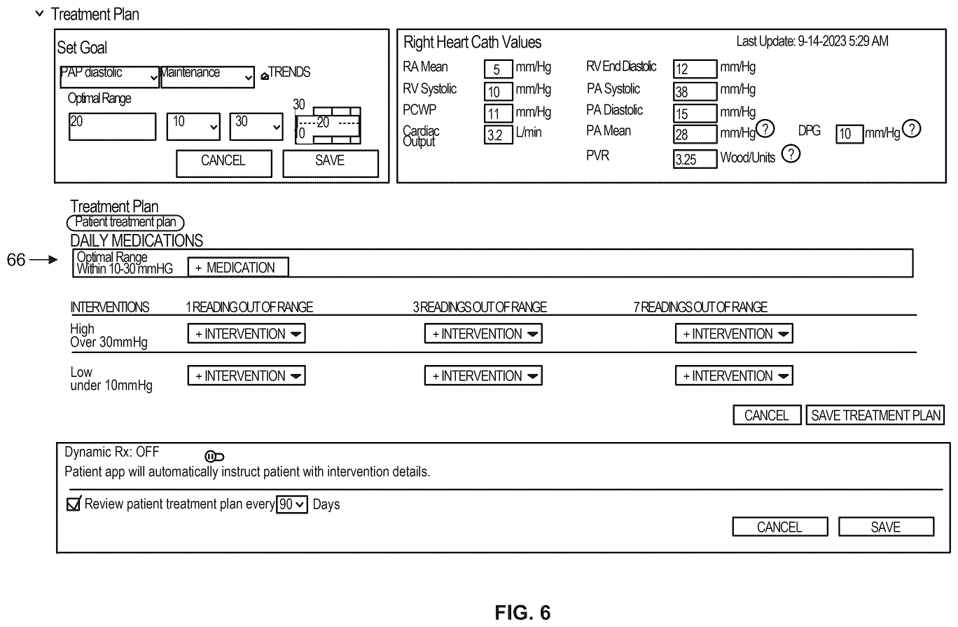

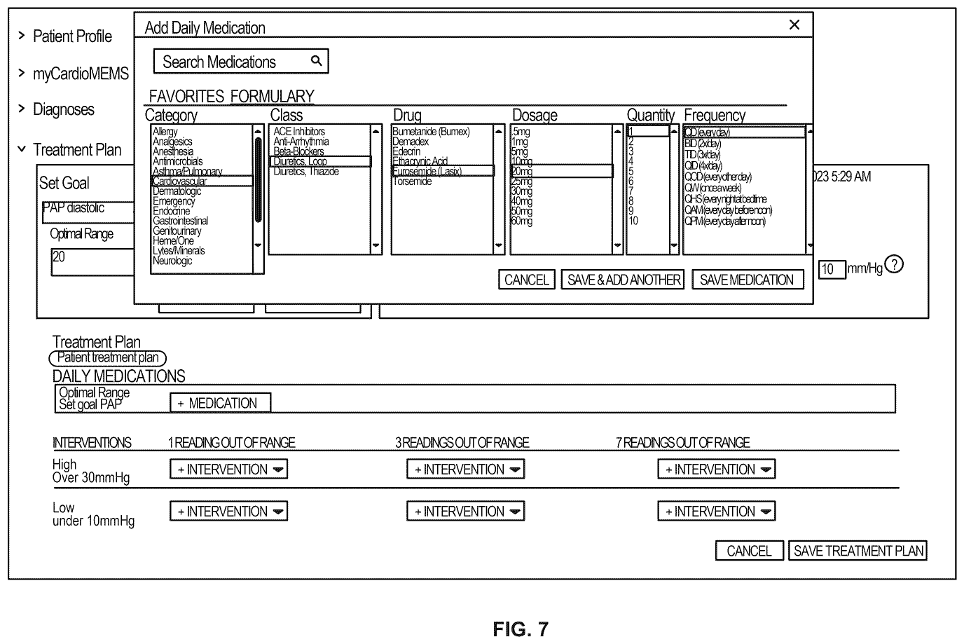

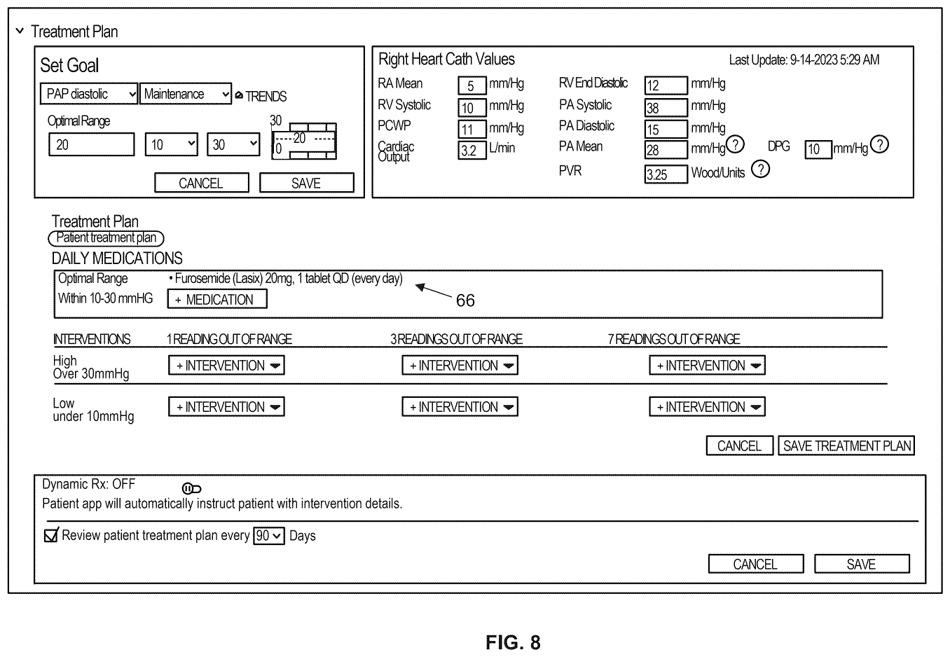

Upon entry of the PAP goal in the PAP parameter section 62, the optimal range for the selected PAP parameter is displayed in the daily medications section 66 as shown in FIG. 6. The daily medications section 66 includes a medications input button that can be selected to access entry forms for specifying daily medications for the patient. Selection of the medications input button accesses the add daily medications entry form shown in FIG. 7, which includes selectable fields for specifying the medication maker, the medication class, the medication, the dosage, the quantity, and the frequency of the selected medication. Any suitable number of daily medications can be added via the add daily medications entry form. Upon entry of the daily medications for the patient, the selected daily medications are displayed in the daily medications section 66 as shown in FIG. 8.

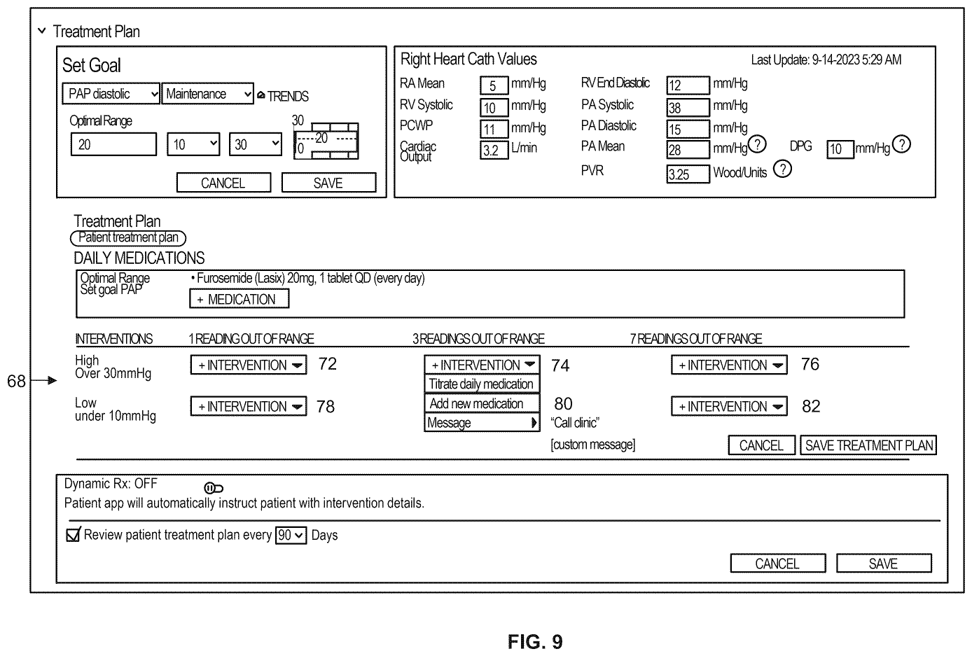

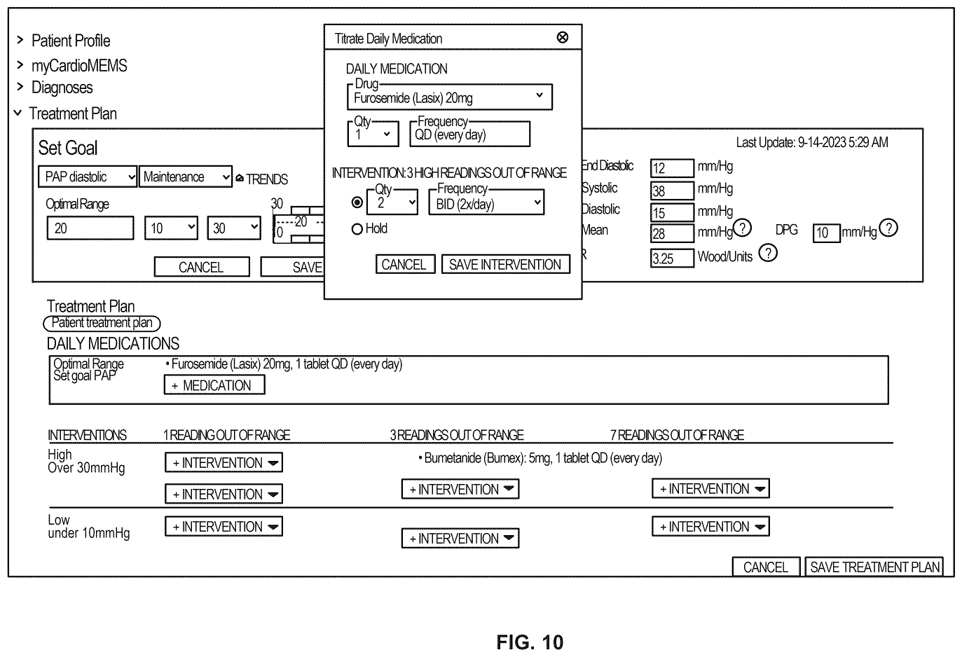

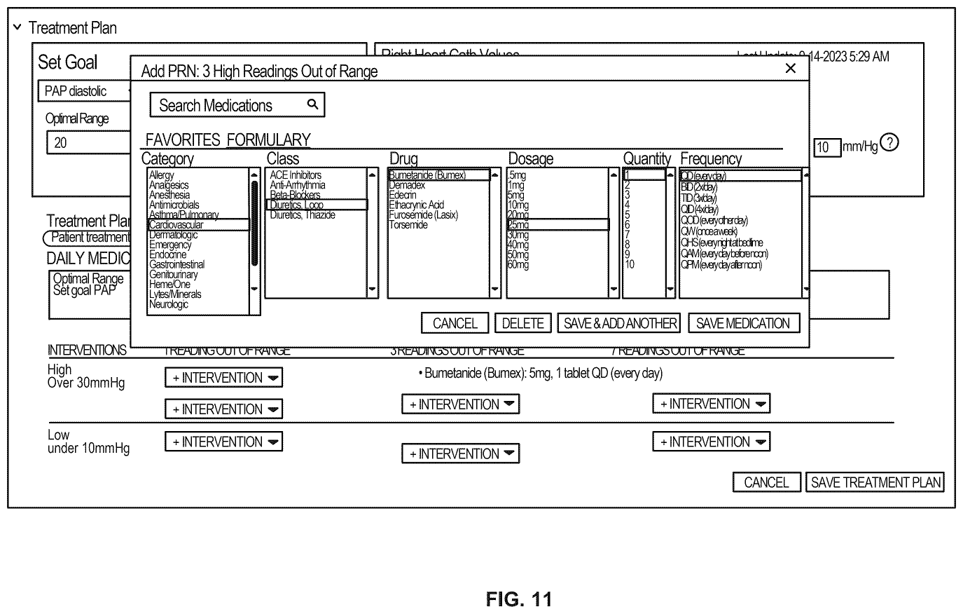

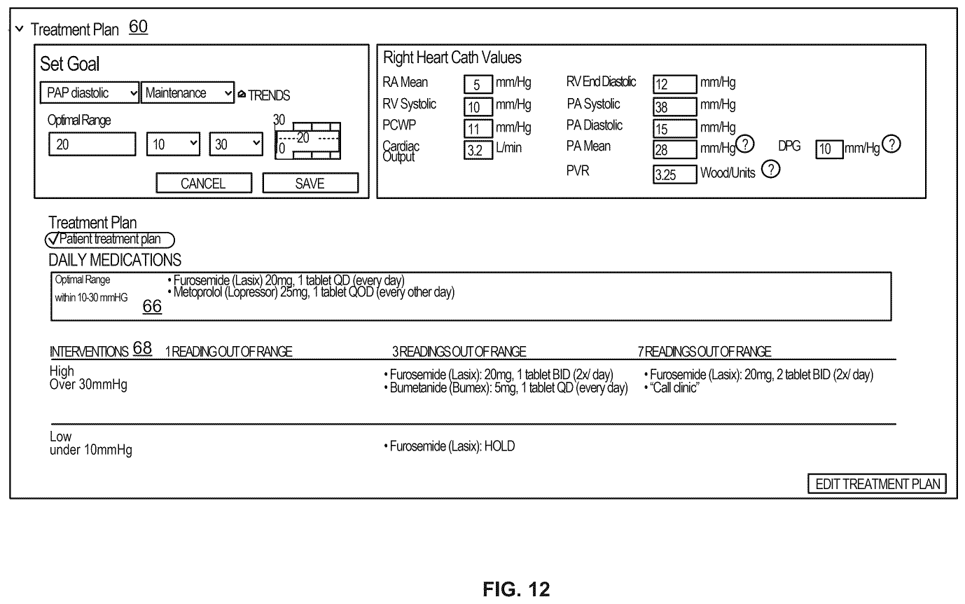

FIG. 9 illustrates entry of interventional treatment instructions via the selectable inputs 72, 74, 76, 78, 80, 82 of the interventional treatment instructions section 68. In the illustrated example, selection of the input 74 (which is used to specify interventional treatment instructions to be automatically transmitted to the patient's mobile application in response to three consecutive PAP parameter readings exceeding the specified upper limit) accesses a list of selectable boxes for selecting whether to titrate the daily medication, add a new medication, or to add a message to be sent the patient. FIG. 10 shows a titrate daily medication entry form that is accessed when the titrate the daily medication option is selected. In the illustrated example, the quantity of the daily medication is increased to 2 per day. FIG. 11 shows an add prescription entry form that is accessed when the add new medication option is selected. The add prescription entry form is structured the same as the add daily medication entry form of FIG. 7. FIG. 12 illustrates the resulting display of specified daily medications and intervention instructions for the patient in the treatment plan section 60.

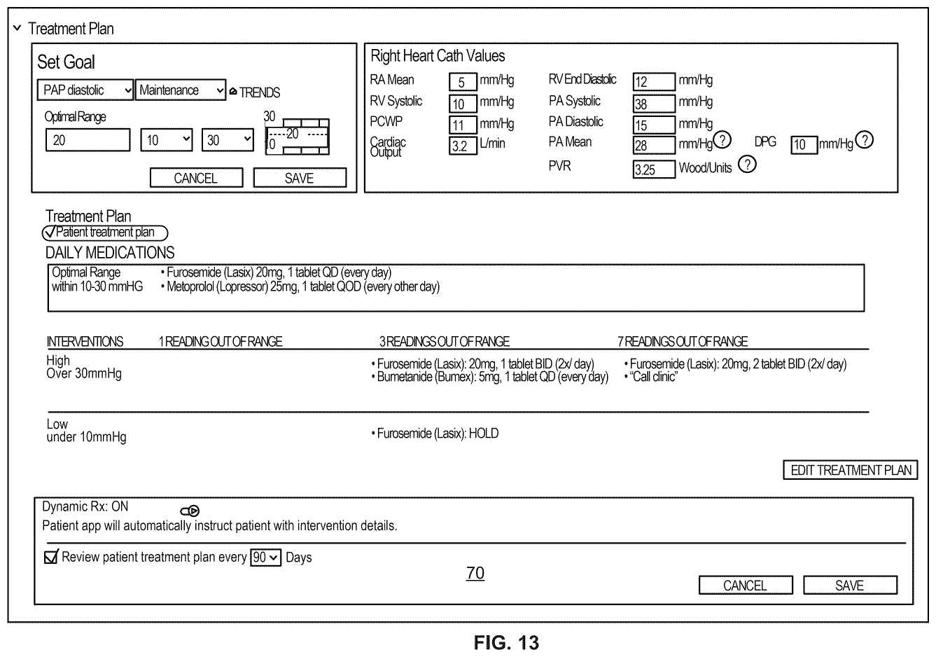

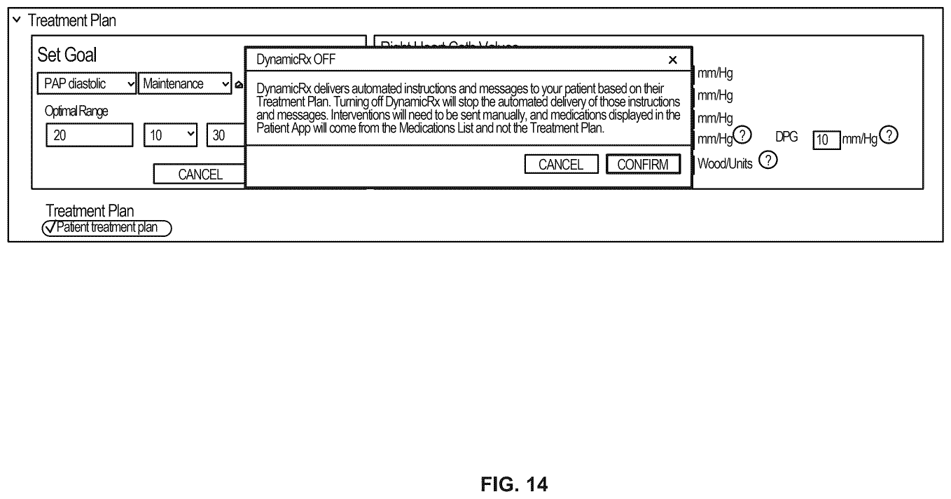

FIG. 13 shows user interface input buttons of the interventional treatment instructions control section 70 that can be selected to save or cancel the specified automated treatment plan for the patient. FIG. 14 shows a confirmation screen that is displayed in response to selection of the cancel input button of the interventional treatment instructions control section 70.

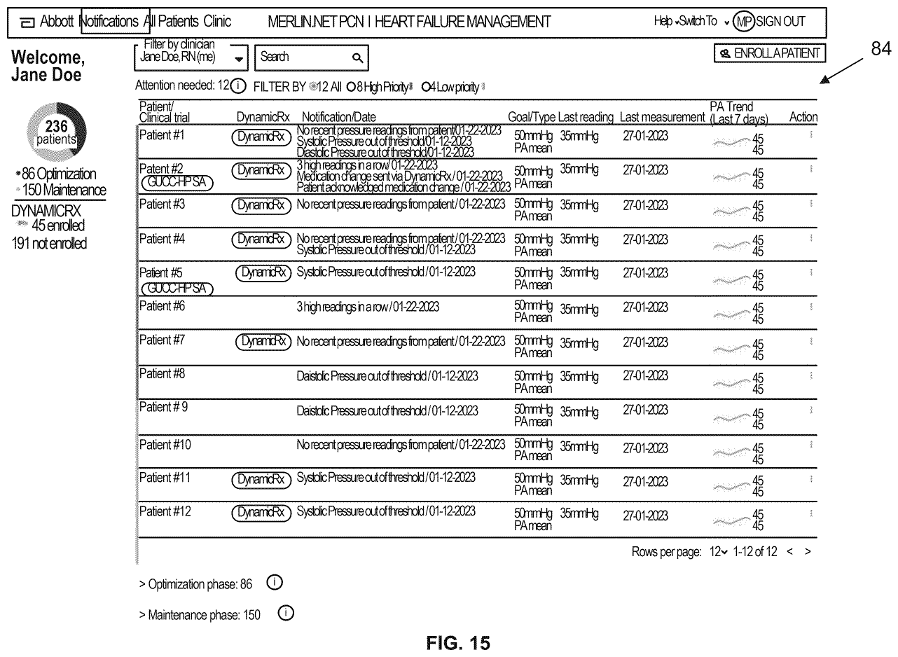

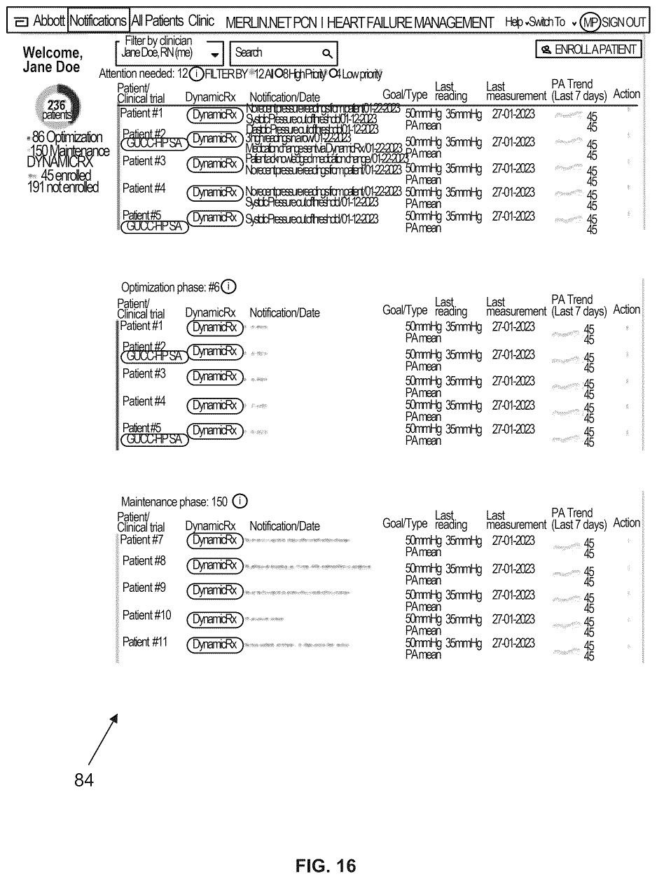

FIG. 15 and FIG. 16 shows a notification screen 84 of the clinician portal 14. The notification screen 84 includes three expandable lists of patients including a list of patients needing attention, a list of patients in an optimization phase, and a list of patients in a maintenance phase.

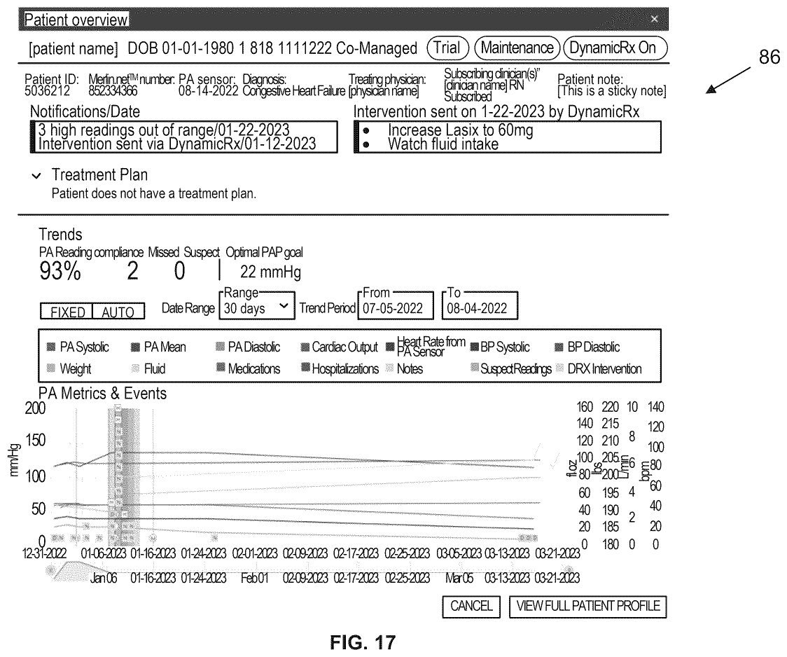

In many embodiments, the clinician portal module is configured to assemble the list of patients on the list of patients needing attention based on pulmonary artery pressure based notification triggering criteria specified by a clinician via the clinician portal 14. For example, the clinician portal module can be configured to receive input via the clinician portal defining a pulmonary artery pressure based notification triggering criteria for the patient, process the pulmonary artery pressure data to determine whether the pulmonary artery pressure based notification triggering criteria is satisfied by the pulmonary artery pressure data, and, in response to determining that the pulmonary artery pressure based notification triggering criteria is satisfied by the pulmonary artery pressure data, place the patient on the list of patients needing attention. In some embodiments, the pulmonary artery pressure based notification triggering criteria is based on one or more exceedances of a notification upper limit for a pulmonary artery pressure parameter for the patient and/or one or more instances of a pulmonary artery pressure parameter for the patient being less than a notification lower limit for the patient. The notification screen 84 displays a prioritized list of patients that can be reviewed by the clinician. Patients who need attention have triggered notifications (previously set by the clinician) based on PA Pressure or other data input. Patients who need attention are pushed to the top of the notifications screen 84. Patients who have not triggered notifications are shown based on whether they are in the optimization phase or the maintenance phase. The clinician can designate each patient to be in the optimization phase or the maintenance phase. The patient can default into the optimization phase upon initial PAP sensor implantation. When the clinician determines that the patient's PAP and therapy have stabilized, the clinician can reclassify the patient into the maintenance phase. Typically, a clinician will review profiles of patients who are in the optimization phase more frequently than profiles of patients who are in the maintenance phase. In the optimization phase, a patient will generally require greater medical management and closer monitoring than a patient in the maintenance phase. In the maintenance phase, additional adjustment of medications (e.g., neurohormonal drugs, vasodilators, and/or diuretics) may be accomplished as needed to maintain or improve pressures, and/or the PADP goal may be re-assessed. FIG. 17 shows a quick overview screen 86 for a selected patient.

### Patient Mobile Application

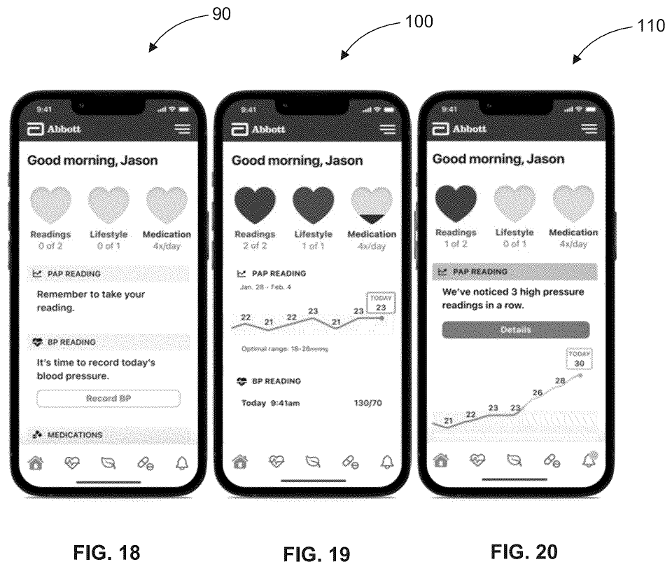

FIG. 18 through FIG. 34 show example user interface screens of the patient mobile application 16. In many embodiments, each of the user interface screens of the patient mobile application 16 are displayed on a display of the mobile device 18 by the patient mobile application 16.

FIG. 18, FIG. 19, and FIG. 20 show example home pages 90, 100, 110 of the patient mobile application 16. Each of the home pages 90, 100, 110 display a reading status indicator of accomplishment of prescribed readings of the PAP sensor 20. In the illustrated embodiment, the reading status indicator is in the form of a reading status icon with a variable appearance for communicating a level of accomplishment of the prescribed readings of the PAP sensor 20. In the illustrated embodiment, the reading status icon has a heart shape. Each of the home pages 90, 100, 110 display a lifestyle status indicator of accomplishment of prescribed lifestyle actions by the patient. In the illustrated embodiment, the lifestyle status indicator is in the form of a lifestyle status icon with a variable appearance for communicating a level of accomplishment of the prescribed lifestyle actions by the patient. In the illustrated embodiment, the lifestyle status icon has a heart shape. Each of the home pages 90, 100, 110 display a medication status indicator of accomplishment of taking prescribed medications by the patient. In the illustrated embodiment, the medication status indicator is in the form of a medication status icon with a variable appearance for communicating a level of accomplishment of taking the prescribed medications by the patient. In the illustrated embodiment, the medication status icon has a heart shape. Each of the home pages 90, 100, 110 display a pulmonary artery pressure reading section. The home pages 90 displays a reminder to accomplish a reading of the pulmonary artery pressure sensor in the pulmonary artery pressure reading section. The home page 100 displays a plot of recent pulmonary artery pressures of the patient in the pulmonary artery pressure reading section. The home page 110 displays a notification regarding recent pulmonary artery pressures of the patient in the pulmonary artery pressure reading section. Each of the home pages 90, 100 display a systemic blood pressure reading section. The home page 90 displays a reminder to measure and enter systemic blood pressure of the patient in the systemic blood pressure reading section. The home page 100 displays an indication of a systemic blood pressure of the patient in the systemic blood pressure reading section.

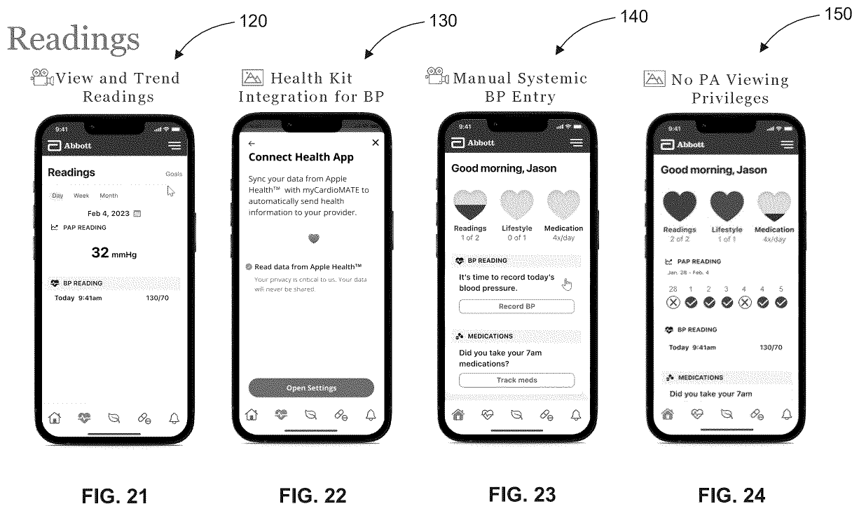

FIG. 21, FIG. 22, FIG. 23, and FIG. 24 show example readings pages 120, 130, 140, 150 for manual and/or automated entry of systemic blood pressure for the patient mobile application 16. The readings page 120 includes a pulmonary artery pressure reading section, a selection button for a day, a selection button for a week, and a selection button for a month. A pulmonary artery pressure of the patient for a day is displayed in the pulmonary artery pressure reading section when the selection button for a day is selected. Pulmonary artery pressures of the patient for a week are displayed in the pulmonary artery pressure reading section when the selection button for a week is selected Pulmonary artery pressures of the patient for a month are displayed in the pulmonary artery pressure reading section when the selection button for a month is selected. The readings page 120 also includes a systemic blood pressure reading section, a selection button for a day, a selection button for a week, and a selection button for a month. A systemic blood pressure reading of the patient for a day is displayed in the systemic blood pressure reading section when the selection button for a day is selected. Systemic blood pressure readings of the patient for a week are displayed in the systemic blood pressure reading section when the selection button for a week is selected. Systemic blood pressure readings of the patient for a month are displayed in the systemic blood pressure reading section when the selection button for a month is selected. The readings page 140 includes the readings icon, the lifestyle icon, the medication icon, a systemic blood pressure entry section, and a medications tracking section. In the illustrated embodiment, the systemic blood pressure entry section displays a reminder to the patient when it is time to enter today's systemic blood pressure measurement. In the illustrated embodiment, the medications tracking section prompts the patient to enter whether the patient has taken prescribed medications.

The readings page 150 includes the readings icon, the lifestyle icon, the medication icon, a PAP reading section, a systemic blood pressure reading section, and the medications tracking section. In the illustrated embodiment, the PAP reading section displays icons indicating whether PAP readings for the previous six days have been accomplished.

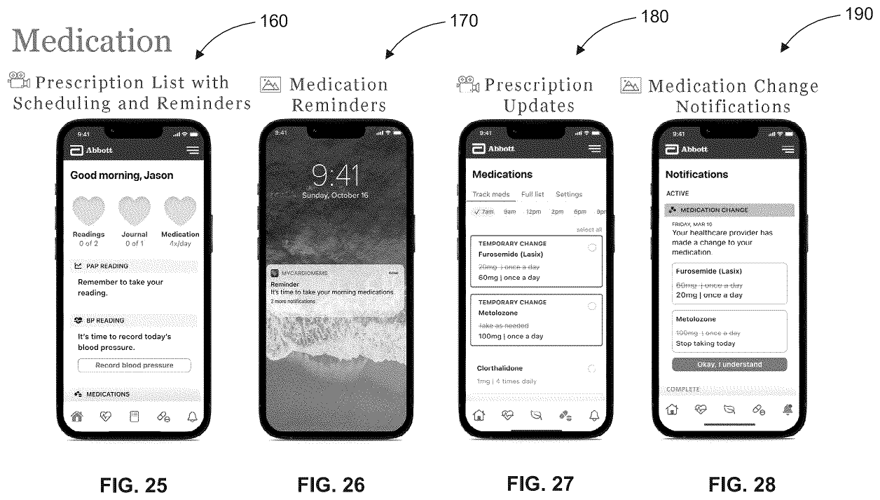

FIG. 25, FIG. 26, FIG. 27, and FIG. 28 show example medications pages 160, 170, 180, 190 for the patient mobile application 16. The medications page 160 includes the readings icon, a journal status indicator icon, the medications icon, the PAP reading section, the systemic blood pressure reading section, and a medication section. The journal status indicator icon indicates status of accomplishment of journal entries by the patient. The medications section includes a list of prescribed medications with scheduling for taking the medications and reminders to take the medications. The medications page 170 displays a reminder to the patient when it is time to take a medication. The medications page 180 informs the patient of prescription updates. The medications page 190 informs the patient of a prescription update(s) and includes an acknowledgement input button that is selectable by the patient to acknowledge the prescription update(s).

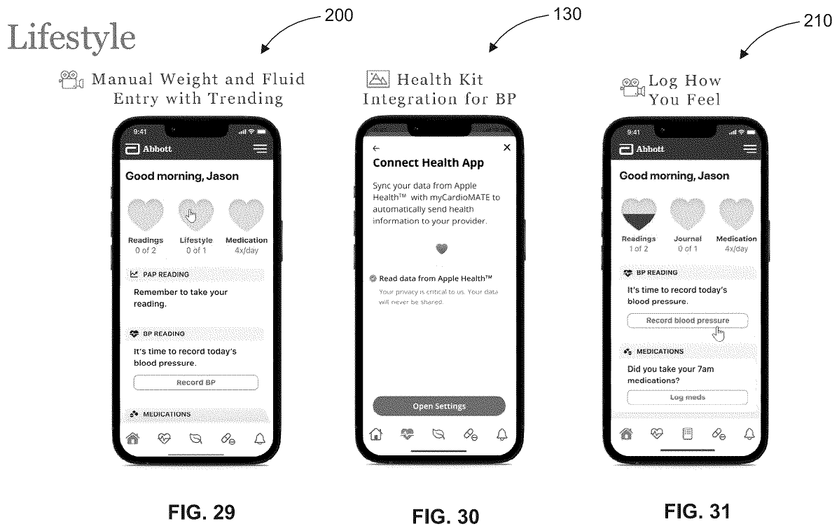

FIG. 29, FIG. 30, and FIG. 31 show example lifestyle pages 200, 130, 210 for the patient mobile application 16. The lifestyle page 200 provides for manual weight entry and fluid intake entry and display of plots of the patient's weight and fluid intake over time. The lifestyle page 210 provides for the input of log entries by the patient.

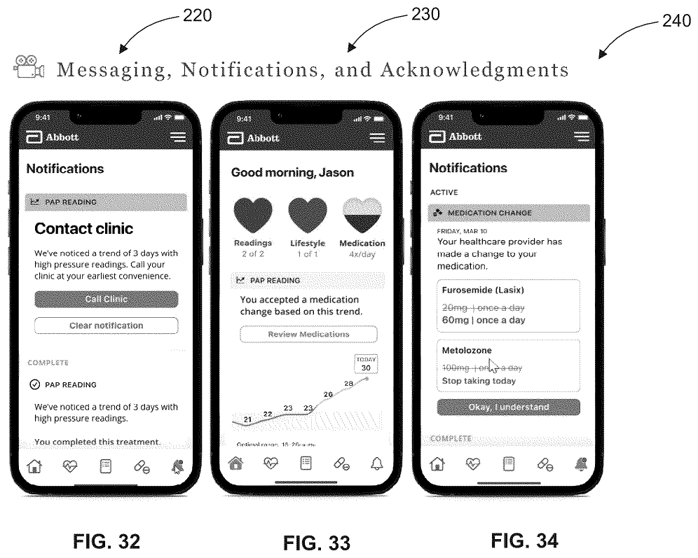

FIG. 32, FIG. 33, and FIG. 34 show example messaging and notification pages 220, 230, 240 for the patient mobile application 16. The page 220 displays a message from the clinic to call the clinic along with a selectable link for calling the clinic. The page 230 notifies the patient regarding patient acceptance of a medication change. The page 240 notifies the patient of a medication change and includes an acknowledgement input button for selection to acknowledge the medication change.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. A system for automated patient management comprising:
at least one processor and at least one memory device storing non-transient instructions executable by the at least one processor to cause the at least one processor to execute a clinician portal module configured to:
receive pulmonary artery pressure data generated via a pulmonary artery pressure sensor implanted in a patient;
receive input via a clinician portal defining a pulmonary artery pressure based interventional treatment triggering criteria for the patient;
receive input, via the clinician portal, defining interventional treatment instructions to be transmitted to the patient in response to the pulmonary artery pressure data satisfying the pulmonary artery pressure based interventional treatment triggering criteria;
process the pulmonary artery pressure data to determine whether the pulmonary artery pressure based interventional treatment triggering criteria is satisfied by the pulmonary artery pressure data; and
in response to determining that the pulmonary artery pressure based interventional treatment triggering criteria is satisfied by the pulmonary artery pressure data, transmit the interventional treatment instructions to a patient mobile application associated with the patient.

2-3. (canceled)

4. The system of claim 1, wherein:
the pulmonary artery pressure based interventional treatment triggering criteria defines an upper limit and a lower limit for a pulmonary artery pressure parameter for the patient;
the interventional treatment instructions comprise high-pressure interventional treatment instructions and low-pressure interventional treatment instructions;
the high-pressure interventional treatment instructions are transmitted to the patient mobile application in response to a designated number of exceedances of the upper limit; and
the low-pressure interventional treatment instructions are transmitted to the patient mobile application in response to a designated number of occurrences of the pulmonary artery pressure parameter being less than the lower limit.

5. The system of claim 1, wherein:
the pulmonary artery pressure based interventional treatment triggering criteria defines an upper limit for a pulmonary artery pressure parameter for the patient;
the interventional treatment instructions comprise first high-pressure interventional treatment instructions and second high-pressure interventional treatment instructions;
the first high-pressure interventional treatment instructions are transmitted to the patient mobile application in response to a first number of consecutive exceedances of the upper limit;
the second high-pressure interventional treatment instructions are transmitted to the patient mobile application in response to a second number of consecutive exceedances of the upper limit; and
the second number of consecutive exceedances of the upper limit is greater than the first number of consecutive exceedances of the upper limit.

6. The system of claim 5, wherein:
the interventional treatment instructions comprise third high-pressure interventional treatment instructions;
the third high-pressure interventional treatment instructions are transmitted to the patient mobile application in response to a third number of consecutive exceedances of the upper limit; and
the third number of consecutive exceedances of the upper limit is greater than the second number of consecutive exceedances of the upper limit.

7. The system of claim 1, wherein:
the pulmonary artery pressure based interventional treatment triggering criteria defines a lower limit for a pulmonary artery pressure parameter for the patient;
the interventional treatment instructions comprise first low-pressure interventional treatment instructions and second low-pressure interventional treatment instructions;
the first low-pressure interventional treatment instructions are transmitted to the patient mobile application in response to a first number of consecutive instances of the pulmonary artery pressure parameter being less than the lower limit;
the second low-pressure interventional treatment instructions are transmitted to the patient mobile application in response to a second number of consecutive instances of the pulmonary artery pressure parameter being less than the lower limit; and
the second number of consecutive instances of the pulmonary artery pressure parameter being less than the lower limit is greater than the first number of consecutive instances of the pulmonary artery pressure parameter being less than the lower limit.

8. The system of claim 7, wherein:
the interventional treatment instructions comprise third high-pressure interventional treatment instructions and third low-pressure interventional treatment instructions;
the third low-pressure interventional treatment instructions are transmitted to the patient mobile application in response to a third number of consecutive instances of the pulmonary artery pressure parameter being less than the lower limit; and
the third number of consecutive instances of the pulmonary artery pressure parameter being less than the lower limit is greater than the second number of consecutive instances of the pulmonary artery pressure parameter being less than the lower limit.

9. The system of claim 1, wherein the clinician portal module is further configured to:
receive input via the clinician portal defining base-line treatment instructions for the patient, wherein the base-line treatment instructions define base-line medications for the patient; and
transmit the base-line treatment instructions to the patient mobile application.

10. The system of claim 1, wherein the interventional treatment instructions comprise a message for the patient.

11. The system of claim 1, wherein the clinician portal module is further configured to, in response to transmitting the interventional treatment instructions to the patient mobile application, display an indication on the clinician portal that the interventional treatment instructions were transmitted to the patient mobile application.

12. The system of claim 1, wherein the clinician portal module is further configured to:
receive an acknowledgement from the patient mobile application that the patient mobile application received an input indicating acknowledgement of receipt of the interventional treatment instructions; and
output an indication of the acknowledgement via the clinician portal.

13. The system of claim 1, wherein the clinician portal module is further configured to:
receive input via the clinician portal defining a pulmonary artery pressure based notification triggering criteria for the patient;
process the pulmonary artery pressure data to determine whether the pulmonary artery pressure based notification triggering criteria is satisfied by the pulmonary artery pressure data; and
in response to determining that the pulmonary artery pressure based notification triggering criteria is satisfied by the pulmonary artery pressure data, output a notification via the clinician portal that designates the patient for assessment.

14.-15. (canceled)

16. The system of claim 1, wherein the patient mobile application is configured for displaying a treatment change notification page comprising the interventional treatment instructions.

17.-19. (canceled)

20. The system of claim 1, wherein the patient mobile application is configured for displaying a medication prescription change notification page comprising an updated medication prescription.

21. The system of claim 1, wherein the patient mobile application is configured to display a status page comprising a reading status indicator of accomplishment of prescribed readings of the pulmonary artery pressure sensor.

22.-23. (canceled)

24. The system of claim 1, wherein the patient mobile application is configured to display a status page comprising a lifestyle status indicator of accomplishment of prescribed lifestyle actions by the patient.

25.-26. (canceled)

27. The system of claim 1, wherein the patient mobile application is configured to display a status page comprising a medication status indicator of accomplishment of taking prescribed medications by the patient.

28.-29. (canceled)

30. The system of claim 1, wherein: the patient mobile application is configured to:
display a status page comprising a pulmonary artery pressure reading section; and
display one of a reminder to accomplish a reading of the pulmonary artery pressure sensor, a plot of recent pulmonary artery pressures of the patient, or a notification regarding recent pulmonary artery pressures of the patient in the pulmonary artery pressure reading section.

31. The system of claim 1, wherein the patient mobile application is configured to:
display a status page comprising a systemic blood pressure reading section; and
display one of a reminder to measure and enter systemic blood pressure of the patient or an indication of a systemic blood pressure of the patient in the systemic blood pressure reading section.

32. The system of claim 1, wherein the patient mobile application is configured to:
display a readings page comprising a pulmonary artery pressure reading section;
display a selection button for a day, a selection button for a week, and a selection button for a month;
display a pulmonary artery pressure of the patient for a day when the selection button for a day is selected;
display pulmonary artery pressures of the patient for a week when the selection button for a week is selected; and
display pulmonary artery pressures of the patient for a month when the selection button for a month is selected.

33. The system of claim 1, wherein the patient mobile application is configured to:
display a readings page comprising a systemic blood pressure reading section;
display a selection button for a day, a selection button for a week, and a selection button for a month;
display a systemic blood pressure reading of the patient for a day when the selection button for a day is selected;
display systemic blood pressure readings of the patient for a week when the selection button for a week is selected; and
display systemic blood pressure readings of the patient for a month when the selection button for a month is selected.
